(19) United States
(12) Patent Application Publication
(10) Pub. No.: US 2011/0300246 A1
(43) Pub. Date: Dec. 8, 2011

(54) Herbal Extracts for Treatment of Diabetic Complications and Oxidative Stress

(76) Inventor: Qinheng Huang, Waltham, MA (US)

(21) Appl. No.: 12/793,319

(22) Filed: Jun. 3, 2010

(51) Int. Cl. A61K 36/42; A61P 3/10; A61K 36/488; A61K 36/68; A61K 36/00

(52) U.S. Cl. 424/758; 424/725; 424/777

(57) ABSTRACT

Compositions, functional foods, methods of preparation, and their uses for the treatment of diabetic complications and oxidative stress are provided. The compositions, functional foods, and methods relate to herbal extracts from the rhizome of Curcuma L., Puerariae radix, the root of herb Pueraria lobat, Fructus corni, the fruit of herb Cornus officinalis Sieb Et Zucc, and herb Plantago astiatice L.

### TECHNICAL FIELD

The present invention relates to the field of pharmacology. More specifically, the invention relates to herbal extracts from Plantago astiatice L, Fructus corni (the fruit of herb Cornus officinalis Sieb Et Zucc), the rhizome of herb Curcuma L., and Puerariae radix (the root of herb Pueraria lobata). The extracts are useful for inhibiting the formation of advanced glycation endproducts (ACEs), for the prevention and treatment of diabetic complications, and also as an anti-oxidant in preventing and attenuating cellular oxidative stress associated with aging.

### BACKGROUND

Diabetes mellitus is the most common chronic disease associated with carbohydrate metabolism and a major cause of disability and the death of people in the 60˜70 year-old age group. More than 220 million people world wide have diabetes currently and this will increase to at least 300 millions by 2025 (WHO, Fact Sheet No. 312, November 2009). Chronic hyperglycemia during the diabetes leads to secondary complications at 10-20 years after the onset of diabetes affecting eyes, kidneys and nerves known as diabetic retinopathy, diabetic nephropathy and diabetic neuropathy.

Non-enzymatic glycosylation of proteins resulting in the accumulation of AGEs has been recognized as a causative factor in the pathogenesis of diabetic complications and diseases of aging [Brownlee M et al, 1988, N Engl J Med 318:1315-1321; Bucalar & Cerami, 1992, Adv Pharm 23:1-34; Singh Ret al, 2001, Diabetologia 44:129-146]. Formation of AGEs involves a complex series of reactions starting with the covalent attachment of carbonyl group of glucose and other reducing sugars to the side-chain amino groups of proteins through nucleophilic addition reaction without the action of enzymes to form Schiff bases [Brownlee M, 1995, Clin Invest Med 18:275-281]. The unstable Schiff base rapidly rearranges to form stable Amadori products, which then undergoes a series of slow reactions involving rearragements and dehydration to form stable, heterogeneous adducts known as AGEs that remain tightly bound to the protein [Zyzak D V et al, 1995, Arch Biochem Biophys 316:547-554; Monnier V M & Cerami A, 1981, Science 211:491-493]. Glucose-derived modification of proteins can also been formed by reactive carbonyl intermediates such as methylglyoxal, glyoxal, 3-deoxyglucosone [LO T W C et al, 1994, J Biol, Chem 269:32299-32305]. Methylglyoxal (MG) has recently received considerable attention as common mediator to form AGEs [Degenhardt T P et al, 1998, Cell Mol Bio 44:1139-1145; Shipanova I N et al, 1997, Arch Biochem Biophys 344:29-36]. In patients with both type I and type II diabetes, the concentration of MG was found to be increased 2˜6 folds [Phillips SA & Thornalley P J., 1993, Eur J Biochem 212:101-105]. Furthermore, MG has also been found not only as the most reactive dicarbonyl AGE-intermediate in cross-linking of proteins, it has been reported to generate reactive oxygen species (ROS) in the course of glycation reactions [Yim et al, 1995, J Biol Chem 270:28228-28233].

AGEs possess highly reactive structures and cause the cross-linking of proteins with —SH and —NH2 groups on adjacent proteins or within domains of the same protein to form irreversible AGE cross-linking [Sajithal G B, et al 1998, Biochem Biophys Acta 1407: 215-224; Fu M-X et al, 1994, Diabetes 43:676-683; Sara Vet al, 2001, Expert Opin Investing Drugs 10:1977-1987]. AGE-derived cross-linking causes proteins that are normally flexible to become rigid, making cells, tissues and blood vessels stiff and increasingly dysfunctional [Luthra M & Balasubramanian D, 1993, J Bio Chem 268: 18119-18127]. In healthy individual, this process occurs naturally and slowly as body ages. In diabetic patients, the rate of AGEs accumulation and the extent of protein cross-linking are accelerated due to the high blood glucose condition. Numerous studies have established the role of AGEs in the development of renal, ophthalmological, neurological and cardiovascular complications in diabetes and aging [Stitt, A W et al, 2002, Expert Opin Investing Drugs 11:1205-1223; Brownlee M., 1994, Diabetes 43:836-841].

Nephropathy is the term applied to a conglomerate of lesions that occur concurrently in the kidney in up to 35-45% of diabetic patients and is a leading cause of end stage renal disease (ESRD) [Krolewski A S et al, 1988, N Engl J Med 318:140-145]. It is characterized by an increased glomerular basement membrane (GBM) thickness, mesangial expansion, glomerulosclerosis and tubulointerstial fibrosis in the late stage [Osterby R, 1986, Clin Endocrinol Metab 15:733-751; Gilbert RE & Cooper M E, 1999, Kidney Int 56: 1627-1637]. Glomerular damage leads to sustained albuminuria and is believed to be due to AGE-induced changes to the glomerular basement membrane [Makita et al, 1991, N Engl J Med 325:836-842; Rajdsc et al, 2000; Am. J Kidney Dis 35:365-380; Vlassera et al, 1994, Proc Natl Acad Sci USA 91:11704-11708]. In animal model of diabetes, immunochemical [Nishino T et al, 1995, Human Pathol 26:308-313; Yamada K et al, 1994, Clin Nephrol 42:354-361] and tissue fluorescence [Soulis-Liparota T et al, 1991, Diabetes 40: 1328-1344] measurements have demonstrated an increased deposition of AGEs in the renal cortex. Accumulation of AGEs eventually leads to the thickening of the GBM along with an increased production of type IV collagen.

Diabetic neuropathy is a very diverse, mutifocal disease that affects 40% of diabetic patients and can cause severe morbidity and premature death [Vinik I et al, 2000, Diabetologia 43:957-973]. It has been proposed that AGEs accumulate in peripheral neurons in diabetic patients and in diabetic animals [Sugimoto K et al, 1997, Diabetologia 40: 1380-1387; Ryle C et al 1997, Muscle nerve 20: 577-584]. The accumulation of AGE crosslinks in the extracellular matrix (ECM) surrounding axons and with Schwann cells have important neuron-specific implications, because growing axons require direct ECM communication for survival and regeneration. It has been suggested that AGE-crosslinks interference with these interaction through depletion of heparin sulfate proteoglycan [Paul R G & Bailey A J, 1999, Int J Biochem Cell Biol 31:53-660].

Diabetic retinopathy is one of the most common microvascular complication of diabetes and the leading cause of new blindness in the working population of developed counties [Aiello L P et al, Diabetes Care 21:143-156] and occurs as a result of long-term accumulated damages to the small blood vessels in the retina. This is mainly a microvascular disease involving proliferation of endothelial cells in capillaries and venues along with widespread pericyte damage. Accumulation of AGEs in retinal vessel wall and AGE's apoptotic effect on pericytes has been proposed as one important mechanism underlying these pathological changes [Hammes H P et al, 1998, Diabetologia 41:165-170; Stitt A W et al, 2000, Mol Cell Biol Res Commun 3: 380-388]. Inhibition of AGE formation using aminoguanidine in animals has been shown to reduce the AGE-specific fluorescence in the retina of diabetic rats and to decrease the severity of microaneurysm and breakdown of the blood-retinal barrier [Hammes HP et al, 1991, Proc Nati Acad Sci USA 88:11555-11588; Kern T S & Engerman R L, 2001, Diabetes 50: 1636-1642; Kern TS, et al 2000, Ivest Ophthalmol Vis Sci 41:3972-3978].

Patients with diabetes also suffer a number of progressive and functional defects in their large vessels culminating in a well-recognized diabetic macroangiopathy that manifest as accelerated coronary artery, cerebrovascular and peripheral vascular disease. In the early stages of diabetes, patients show increased arterial stiffness [Taniwaki H et al, 2001, Atherosclerosis 158: 207-214], and reduced vascular compliance [Romney J S & Lewanczuk R Z., 2001, Diabetes care 24:2102-2106]. AGEs are now recognized as having a significant role in the formation and progression of atherosclerotic lesions [Stitt A W et al, 1997, Ann NY Acad Sci 811:115-127]. Many studies have shown increased AGEs in the macrovasculature during diabetes and these adducts provoke pathogenic effects on the incumbent endothelial cells, macrophages and smooth muscle cells as atherosclerotic form [Horiuchi S et al, 1996, Diabetes 45(supp1.3):S73-S76; Sakata N et al, 1998, Atherosclerosis 141: 61-75; Stitt A W et al, 1997, Mol Med 3:617-627].

As described above, the roles of AGEs in the pathological squeal of diabetes and aging indicates that the inhibition of AGEs formation and accumulation in the tissues is potential therapeutic strategy to prevent and treat diabetic complications.

To date, the most extensively investigated inhibitor of AGEs formation and AGE-protein cross-linking is the small nucleophilic hydrazine compound aminoguanidine (pimagedine). This prototype AGE inhibitor has been shown to prevent a range of vascular complications in experimental diabetic animals [Abdel-Rahman & Bolton WK, 2002, Expert Opin Investing drugs 11:565-574; Yamauchi A et al, 1997, Diabetes Res Clin Prac 34: 127-133; Soulis-Liparota T et al, 1991, Diabetes 40: 1328-1334], and achieved significant lowering of urinary albumin and showed some retardation of the progression of nephropathy and retinopathy in multi-centre clinical trials [Appel et al, 1999, J Am Soc Nephrol 10:153A; Whittier F et al., 1999, J Am Soc Nephrol 10:184A; Abdel-Rahman & Bolton, 2002]. Unfortunately, aminoguanidine possesses severe side effects such as flu-like syndrome, anemia, increased antinuclear autoantibodies, glomerulonephritis, upper GI symptoms and congestive heart failure, which exclude its clinic use. Thus, development of safer anti-AGE drugs is urgently needed.

### SUMMARY OF THE INVENTION

This section is for the purpose of summarizing some aspects of the present invention and to briefly introduce some preferred embodiments. Simplifications or omissions in this section as well as in the abstract may be made to avoid obscuring the purpose of this section and the abstract. Such simplifications or omissions are not intended to limit the scope of the present invention.

The present inventor has found that herbal extracts from Plantago astiatice L, Fructus corni, Curcuma L. rhizome, and Pueraria radix have excellent inhibitory activity against the formation and accumulation of AGEs and were also found to exhibit anti-oxidant properties. Mixtures of the herbal extracts were surprisingly found to have inhibitory AGE activity superior to that of any single herbal extract and aminoguanidine. Methods of isolating the herbal extracts, compositions and functional foods comprising the extracts, and their uses are provided by the present invention, and are further described in the drawings and detailed description that follow.

Other objects, features, benefits and advantages, together with the foregoing, are attained in the exercise of the invention in the following description and resulting in the embodiment illustrated in the accompanying drawings.

### BRIEF DESCRIPTION OF THE DRAWINGS

These and other features, aspects, and advantages of the present invention will be better understood with regard to the following description, appended claims, and accompanying drawings where:

FIG. 1 shows graphically the in vivo blood glucose-lowering effect of the inventive herbal mixture in STZ-induced diabetic rats during the course of 12 weeks treatment. NC+CMC: normal group+carboxymethylcellulose; DC+CMC: diabetes-induced group+carboxymethylcellulose; DC+AG: diabetes-induced group+positive control aminogunidine; DC+HEM: diabetes-induced group+herbal extract mixture; All data are expressed as mean±SD of 8 animals for each group. * P<0.05, ** P<0.01 versus DC+CMC group and DC+AG group by one-way ANOVA analysis followed by Bonferroni's post hoc test.

FIG. 2 shows graphically effect of the inventive herbal mixture on the immunohistochemical staining for insulin in the pancreatic islets of the rats. DC+CMC: diabetes-induced group+carboxymethylcellulose; DC+AG: diabetes-induced group+positive control aminogunidine; DC+HEM: diabetes-induced group+herbal extract mixture;

FIG. 3 shows graphically the in vivo inhibitory effect of the inventive herbal extract mixture on the levels of AGE-fluorescence in the pancreas of STZ-induced diabetic rats. NC+CMC: normal group+carboxymethylcellulose; DC+CMC: diabetes-induced group+carboxymethylcellulose; DC+AG: diabetes-induced group+positive control aminogunidine; DC+HEM: diabetes-induced group+herbal extract mixture. All data are expressed as mean±SD of 8 animals for each group. *** P<0.001 versus NC+CMC group; ## P<0.01 versus DC+CMC group by one-way ANOVA analysis followed by Bonferroni's post hoc test.

FIG. 4 shows graphically the in vivo inhibitory effect of the inventive extract mixture on the levels of AGE-fluorescence in the lenses of STZ-induced diabetic rats. NC+CMC: normal group+carboxymethylcellulose; DC+CMC: diabetes-induced group+carboxymethylcellulose; DC+AG: diabetes-induced group+positive control aminogunidine; DC+HEM: diabetes-induced group+herbal extract mixture. All data are expressed as mean±SD of 8 animals for each group. *** P<0.001 versus NC+CMC group; # P<0.05, ## P<0.01 versus DC+CMC group by one-way ANOVA analysis followed by Bonferroni's post hoc test.

FIG. 5 shows graphically the in vivo inhibitory effect of the inventive herbal extract mixture on the levels of CML-AGE in the lenses of STZ-induced diabetic rats. NC+CMC: normal group+carboxymethylcellulose; DC+CMC: diabetes-induce carboxymethylcellulose; DC+AG: diabetes-induced group+positive control aminogunidine; DC+HEM: diabetes-induced group+herbal extract mixture. All data are expressed as mean±SD of 8 animals for each group. * P<0.05, *** P<0.001 versus NC+CMC group; ## P<0.01 versus DC+CMC group by one-way ANOVA analysis followed by Bonferroni's post hoc test.

FIG. 6 shows graphically the in vivo inhibitory effect of the inventive herbal extract mixture on the levels of AGE-fluorescence in the descending aorta of STZ-induced diabetic rats. NC+CMC: normal group+carboxymethylcellulose; DC+CMC: diabetes-induced group+carboxymethylcellulose; DC+AG: diabetes-induced group+positive control aminogunidine; DC+HEM: diabetes-induced group+herbal extract mixture. All data are expressed as mean±SD of 8 animals for each group. * P<0.05, *** P<0.001 versus NC+CMC group; ## P<0.01, ### P<0.001 versus DC+CMC group by one-way ANOVA analysis followed by Bonferroni's post hoc test.

FIG. 7 shows graphically the in vivo inhibitory effect of the inventive extract mixture on the levels of CML-AGE in the descending aorta of STZ-induced diabetic rats. NC+CMC: normal group+carboxymethylcellulose; DC+CMC: diabetes-induce carboxymethylcellulose; DC+AG: diabetes-induced group+positive control aminogunidine; DC+HME: diabetes-induced group+herbal extract mixture. All data are expressed as mean±SD of 8 animals for each group. *** P<0.001 versus NC+CMC group; ## P<0.01, ### P<0.001 versus DC+CMC group by one-way ANOVA analysis followed by Bonferroni's post hoc test.

FIG. 8 shows graphically the in vivo inhibitory effect of the inventive extract mixture on urinary albumin excretion in STZ-induced diabetic rats. NC+CMC: normal group+carboxymethylcellulose; DC+CMC: diabetes-induce carboxymethylcellulose; DC+AG: diabetes-induced group+positive control aminogunidine; DC+HEM: diabetes-induced group+herbal extract mixture. All data are expressed as mean±SD of 8 animals for each group. *** P<0.001 versus NC+CMC group; ## P<0.01 versus DC+CMC group by one-way ANOVA analysis followed by Bonferroni's post hoc test.

### DETAILED DESCRIPTION OF THE INVENTION

The detailed description of the invention is presented largely in terms of procedures, steps, logic blocks, processing, and other symbolic representations. These process descriptions and representations are typically used by those skilled in the art to most effectively convey the substance of their work to others skilled in the art. Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams, if any, representing one or more embodiments of the invention do not inherently indicate any particular order nor imply any limitations in the invention.

In one embodiment, provided is a pharmaceutical composition comprising a mixture of herbal extracts from each of Plantago astiatice L, Fructus corni, Curcuma L. rhizome, and Puerariae radix. In some aspects of the composition, the herbal extracts from each of said Plantago astiatice L, Fructus corni, Curcuma L. rhizome, and Puerariae radix is present in an amount that is 5-85% by weight based on 100% by weight of the total mixture of extracts.

In another embodiment, provided is a functional food comprising a mixture of herbal extracts from each of Plantago astiatice L, Fructus corni, Curcuma L. rhizome, and Puerariae radix. In some aspects of the functional food, the herbal extracts from each of said Plantago astiatice L, Fructus corni, Curcuma L. rhizome, and Puerariae radix is present in an amount that is 5-85% by weight based on 100% by weight of the total mixture of extracts.

In another embodiment, provided is a method for inhibiting formation of advanced glycation endproducts in a subject in need thereof comprising administering to the subject a composition or functional food as described herein. In another embodiment, provided is a method for preventing or treating a diabetic complication or oxidative stress in a subject in need thereof comprising administering to the subject a composition or functional food as described herein. In some aspects, the diabetic complication is selected from the group consisting of diabetic nephropathy, diabetic neuropathy, and diabetic retinopathy.

In other embodiments, provided is a method of isolating herbal extracts from Plantago astiatice L, the method comprising boiling powdered Plantago astiatice L in an aqueous solution; filtering and concentrating the aqueous solution; and adding anhydrous ethanol to precipitate the herbal extracts. In some aspects, provided are herbal extracts of Plantago astiatice L isolated by the methods as described herein.

In other embodiments, provided is a method of isolating herbal extracts from Fructus corni, the method comprising refluxing powdered Fructus corni in absolute methanol solution filtering and concentrating the solution to form a concentrate; suspending the concentrate in water to form an aqueous solution; washing the aqueous solution with hexane and ethyl acetate; and lyophilizing the aqueous solution to isolate the herbal extracts. In some aspects, provided are herbal extracts of Plantago astiatice L isolated by the methods as described herein.

In other embodiments, provided is a method of isolating herbal extracts from Curcuma L. rhizome, the method comprising refluxing powdered Curcuma L. rhizome in ethanol; and filtering and concentrating the ethanol yield the herbal extracts. In some aspects, provided are herbal extracts of Curcuma L. rhizome isolated by the methods as described herein.

In other embodiments, provided is a method of isolating herbal extracts from Puerariae radix, the method comprising extracting powered Puerariae radix with ethanol; filtering and concentrating the ethanol to yield a concentrate; suspending the concentrate in water to form an aqueous solution; washing the aqueous solution with ethyl acetate; and lyophilizing the aqueous solution to isolate the herbal extracts. In some aspects, provided are herbal extracts of Puerariae radix isolated by the methods as described herein.

In some aspects of the methods provided herein, the herbal extracts from Plantago astiatice L, Fructus corni, Curcuma L. rhizome, and Puerariae radix are further purified on a polymeric resin column such as a Diaion ion exchange resin column.

In other embodiments, provided is a pharmaceutical composition or functional food comprising a mixture of herbal extracts from each of Plantago astiatice L, Fructus corni, Curcuma L. rhizome, and Puerariae radix, wherein at least one of the herbal extracts are isolated according to the methods provided herein.

In one embodiment, provided are methods for the preparation of an active extract from Plantago astiatice L, Fructus corni, Curcuma L. rhizome and Puerariae radix. The “active” referred here meant the activity of inhibiting the formation of AGEs.

In one embodiment, provided is a composition for the prevention and treatment of diabetic complication and aging by inhibiting the formation and accumulation of AGEs, which contains a mixture of herbal extracts from Plantago astiatice L, Fructus corni, Curcuma L. rhizome and Puerariae radix as active ingredients.

In one embodiment, provided is a pharmaceutical composition for the prevention and treatment of diabetic complication, which contains the said herbal extract mixture as active ingredients.

In one embodiment, provided is a functional food for the prevention and treatment of diabetic complication, which contains the said single herbal extract or the said herbal extract mixture as active ingredients.

In one embodiment, provided is a pharmaceutical composition for the prevention and delay of aging, which contains, as active ingredients, the said single herbal extract or the said herbal extract mixture.

In one embodiment, provided is a functional food for the prevention and delay of aging, which contains, as active ingredients, the said single extract, or the said medicinal herbal mixture.

In one embodiment, the present invention provides method for preparation of an active extract from Plantago astiatice by the following steps: (1) crushing and grinding the dried herb material into powder; (2) extracting the dried herbal material with aqueous solution at boiling temperature; (3) filtering and concentrating the filtrate under reduced pressure; (4) the resulting concentrated extract was then precipitated by adding anhydrous ethanol and the precipitates were centrifuged, washed with anhydrous ethanol and dried at 40° C. yielding a crude extract; (5) the crude extract was dissolved in H₂O and applied to diaion HP-20 column and eluted with H₂O/MeOH at 100:0 and 0:100, respectively. The MeOH eluents were collected and lyophilized yielding the said active extract.

In another embodiment, the present invention provides method for preparation of an active extract from Fructus corni by the followings: The powdered herbal material was extract in absolute methanol (MeOH) to exhaustion under reflux at 65° C. Extracts were then filtered and concentrated in vacuum and resuspended in H₂O. The resulting aqueous solution was then partitioned sequentially with hexane and ethyl acetate (EtOAc). The resulting water layer was lyophilized and the lyophilized extract was then re-suspended in H₂O and subjected to diaion HP-20SS column and eluted with H₂O/MeOH at 100:0→50:50→100:0 sequentially. The fractions of H₂O/MeOH (50:50) were collected and lyophilized yielding the said active extract.

In still another embodiment, the present invention provides method for preparation of an extract from Puerariae radix by the following steps: The powdered Puerariae radix materials were extracted three times with 80% ethanol at room temperature for 72 hrs each time under the constant stirring condition. The combined extracts were then filtered and concentrated in vacuum and resuspended in H₂O. The resulting aqueous solution was then partitioned with ethyl acetate (EtOAc). The resulting water layer was lyophilized and the lyophilized extract was then re-suspended in H₂O and subjected to diaion HP-20SS column and eluted with H₂O/MeOH at 100:0 and 0:100, respectively. The MeOH elutes were collected and lyophilized yielding the said active extract.

In still another embodiment, the present invention provides method for preparation of an active extract from Curcuma L. rhizome by the following steps: The powdered Curcuma L. rhizome were extracted with 100% ethanol to exhaustion under reflux at 65° C., and the extracts were filtered, concentrated and dried in vacuum at reduced pressure yielding the side active extract.

In still another embodiment, the present invention provides a composition containing a mixture of extracts from Plantago astiatice, Fructus corni, Curcuma L. rhizome and Puerariae radix, in which the amount of each extracts is 5-85% by weight based on 100% by weight of the mixture of extracts.

### Herbal Materials

Plantago astiatice is a perennial plant that belongs to the plantaginaceae family, and a traditional herbal medicine in Asia used for antihypertension, diuretic, antitussive, expectorant, and antiphlogistic purposes [Commission of Chinese Pharmacopoeia, 2005; Galisteo M et al., 2005, J Nutr 135: 2399-2404; Wang S M et al., 2006, Chin J Clin Rehab 10: 184-186]. It has also been reported that this plant possesses a broad-spectrum of antiviral, anticancer, immunomodulation and antidepressant effects [Chiang LC et al 2003, Am J Chin Med 31:225-234; Xu C et al, 2004, J Ethnopharmacol 91:345-349]

Fructus corni is the fruit of herb Cornus officinalis Sieb. Et ZUcc, and is commonly prescribed in Traditional Chinese Medicine (TCM) as a tonic formula and considered to possess actions including invigoration of the liver and kidney, preservation of essence, immunomodulation [Guo LL et al, 2001, Chin J Beijing Univ TCM 24:38-40; Mathad V T et al, 1998, Bioorg Med Chem 6: 605-611], anti-inflammation, anti-shock [Wang TS et al, 1999, Chin J Nanjing Univ TCM 15:345-346], anti-diabetes [Yamahara J et al., 1981, Yakugaku Zasshi 101: 86-90; Jayaprakasam B et al, 2005, J Agri Food Chem 53:28-31; Chen CC et al, 2008, J Ethnopharmacol 117:483-490] and cognitive-enhancing activities [Lee K Y et al, 2009, Arch Pharm 32:677-683].

Curcuma L. rhizome has a long history of use in traditional medicines of China and India [Ammon H et al, 1991, Planta Med 57:1-7]. The polyphenol curcumin is the active ingredient in the herbal remedy and dietary spice turmeric. Curcumin has a surprisingly wide range of beneficial properties, including anti-inflammatory, antioxidant, chemopreventive and chemotherapeutic activity [Aggarwal BB et al, 2007, Adv Exp Med Biol 595: 1-75]. The pleiotropic activities of curcumin derive from its complex chemistry as well as its ability to influence multiple signaling pathways, including survival pathways such as those regulated by NF-kB, Akt, and growth factors; cytoprotective pathways dependent on Nrf2; and metastatic and angiogenic pathways [Hatcher H et al, 2008, Cell Mol Life Sci 65:1631-1652]. Curcumin is a free radical scavenger and exhibits antioxidant activity [Sharma OP., 1976, Biochem Pharmacol 25:1811-1812]. Curcumin is remarkably non-toxic and exhibits great promise as a therapeutic agent, and is currently in human clinical trials for a variety of conditions, including multiple myeloma, pancreatic cancer, myelodysplastic syndromes, colon cancer, psoriasis and Alzheimer's disease[Jurenka J J., 2009, Altern Med Rev 14:141-153].

Puerariae radix is the root of a wild leguminous creeper, Pueraria lobata (Willd) Ohwi and is one of the most popular drugs in Traditional Chinese Medicine. Puerariae radix is commonly known as “Ge-Gen” in China and as Kudzu in Japan. The use of Ge-gen decotion is recorded in the classical medical book Shang Han Lun (“Treatise on fever”), composed more than 1800 years ago. The reported pharmacological actions of Puerariae radix include fever alleviation, blood pressure lowering, memory enhancement, cerebral blood flow increase, coronary artery dilatation, heart function improvement, antiarrhythmic actions and the like. Nowadays it is mainly known as a treatment for alcohol abuse (antidipsotropic agent) [Keung W M & Vallee B L., 1998, Phytochemistry 47: 499-506].

As used herein, the term “subject” refers to mammals including humans, non-human mammals such as dogs and cats, and laboratory animals such as mice, rat, and rabbits, the term “preventing” or “prevention” of a disease in a subject refers to preventing the disease from occurring in a subject that is predisposed or does not yet display symptoms of the disease, the term “treating” or “treatment” of a disease in a subject refers to 1) inhibiting the disease or arresting its development; or 2) ameliorating or causing regression of the disease, and the term “oxidative stress” refers to cellular damage caused by reactive oxygen species (ROS).

Reactive oxygen species are reduced forms of oxygen and include but are not limited to radical and non-radical oxygen species such as superoxide (O₂), hydroxyl radical (HO), hydrogen peroxide (H₂O₂), and lipid peroxides. Cellular oxidative stress contributes to aging via a number of mechanisms such as oxidation of cellular components leading to impairment of normal cellular function.

In one embodiment, provided is a composition for the prevention and treatment of diabetic complication and aging contains, as active ingredients, a mixture of herbal extracts obtained by the steps of: (1) extracting active ingredients from Plantago astiatice herbal materials as described in Example 1 below; (2) extracting active ingredients from Fructus corni herbal materials as described in Example 2 below; (3) extracting active ingredients from Puerariae radix herbal materials as described in Example 3 below; (4) extracting active ingredients from Curcuma L. rhizome herbal materials in Example 4 below; and (5) mixing the extracts of the four herbal materials obtained in the above mentioned steps 1 to 4, the amount of each of the herbal extracts being 5-85% by weight based on the total weight of the herbal extract mixture taken as 100% by weight.

In vitro tests, the inventive single herbal extract and the herbal extract mixture effectively inhibited the formation of AGEs. In experimental diabetic animal model test, a composite of the inventive herbal extract mixture significantly lowered blood glucose; improved the diabetic animals' general well-being; reduced the levels of AGEs in the kidney, pancreas, eye lens and descending aorta of diabetic animals. Furthermore, the herbal extract mixture showed a remarkable reduction in the levels of urinary albumin excretion, a marker of progression of diabetic nephropathy. In addition, the herbal extract mixture also exhibited an excellent antioxidant effect evidenced by increasing the activity of renal superoxide dismutase, a major endogenous anti-oxidative defense enzyme; reducing the levels of renal malondialdehade (MDA), a biomarker of lipid peroxidation.

Accordingly, the inventive single herbal extract, the composite of mixed herbal extracts will be useful for the prevention and treatment of diabetic complications caused by the formation and accumulation of AGEs, for example, diabetic retinopathy, diabetic cataract, diabetic nephropathy, and diabetic neuropathy, etc.

In clinical administration, the inventive single herb extract, mixed herbal extract compositions may be administered by various routes at effective amounts, and used as a general drug formulation.

The composition also contains a pharmaceutically acceptable carrier. Any pharmaceutically acceptable carrier may be used if it is the standard pharmaceutical carrier which can be used in known formulations, such as sterilized solutions, tablets, coated tablets, and capsules. Generally, carriers include excipients, such as starch, milk, sugar, particular clays, gelatin, stearic acid, talc, vegetable oil, gum, glycol, and other known excipients. In addition, sweetening agents, pigment additives, and other components may also be included.

The inventive composition containing the inventive single herbal extract or mixed herbal extracts as an active ingredient may be administered by various routes including but not limited to, oral, intravenous, intramuscular, transdermal routes, etc. In actual clinical administration, the inventive composition may be administered orally or parenterally in the form of various formulations, and it is formulated with generally used diluents or excipients, such as fillers, vehicles, binders, wettings, disintegrants, surfactants, etc.

Solid formulations for oral administration include tablets, pills, powders, granules, capsules and the like, and these solid formulations are prepared with at least one excipient, such as starch, calcium carbonate, sucrose, lactose, gelatin, etc. In addition to simple excipients, lubricants such as magnesium stearate and talc may also be used.

Liquid formulations for oral administration include suspensions, internal dosage forms, emulsions, syrups, etc., in which case simple diluents, such as water or liquid paraffin, and other various excipients, such as wetting agents, sweetening agents, aromatics, and preservatives, may be used.

Furthermore, the inventive composition may be administered by parenteral routes, such as subcutaneous, intravenous and intramuscular injections. In order to prepare formulations for parenteral administration, the single herbal extract or the mixed herbal extract is mixed with a stabilizer or buffer in water to form a solution or suspension, which is then formulated into unit-dose ampoules or vials.

The dose of the inventive single herbal extract or mixed herbal extract is suitably selected depending on the in vivo absorption, inactivation rate and excretion rate of the active ingredient, the age, sex and condition of patients, and the severity of diseases to be treated. It may be administered 1-4 times daily. The accurate dose, administration route and number of the inventive formulation can be easily determined depending on the properties of the formulation, the bodyweight and condition of a subject, and the properties of particular derivatives to be used.

The inventive single herbal extract or mixed herbal extract may be used for the prevention and treatment of diabetic complications and for the prevention and delay of aging, alone or in combination with other known therapeutic agents.

The inventive single herbal extract or mixed herbal extract may be added to foods for improving diseases caused by diabetic complication and aging. The inventive single herbal extract or mixed herbal extract may be added to foods alone or in combination with other foods or food additives, and suitably used according to a conventional method.

The amount of addition of the active ingredient (herbal extract) can be suitably determined depending on the use purpose (prevention, health promotion or treatment). Generally, in the preparation of foods or drinks, the inventive herbal extract mixture is added at an amount of less than 10% by weight, and preferably 3-5% by weight, based on 100% of raw materials. However, for the purpose of health and hygiene or in the case of long-term intake for health control, the amount of addition of the inventive extract may be lower than the above-described amount. However, since the extract has no problem in view of safety, the active ingredient may also be used at a higher amount than the above-described amount.

There are no special limitations on the kind of the foods. Examples of foods to which the extract may be added include dairy products, such as meats, sausages, breads, chocolates, candies, snacks, confectioneries, pizzas, noodles, fried noodles, gums and ice creams, various soups, beverages, teas, drinks, alcoholic drinks and vitamin complexes, as well as all foods which are functional in a common sense.

The inventive functional foods may additionally contain various sweetening agents or natural carbohydrates as in conventional beverages. The natural carbohydrates include monosaccharides, such as glucose and fructose, disaccharides, such as maltose and sucrose, polysaccharides, such as dextrin and cycodextrin, and sugar alcohols, such as xylitol, sorbitol, and erythritol. Sweeteners include natural sweeteners such as thaumatin and stevia extracts, and synthetic sweeteners, such as saccharin and aspartame.

In addition, the inventive composition may contain various nutrients, vitamins, electrolytes, flavoring agents, colorants, pectic acid or its salt, alginic acid or its salt, organic acids, protective colloidal tackifiers, pH adjusters, stabilizers, preservatives, glycerin, alcohol, carbonating agents used in carbonated drinks, etc. Also, the inventive composition may contain fruit flesh for the preparation of natural fruit juices, fruit juice beverages and vegetable juices. These components may be used alone or in combination.

The present invention will be described in further detail by the following examples. It is to be understood, however, that these examples are given for illustrative purpose only and are not construed to limit the scope of the present invention.

### Example 1

### Preparation of the Active Extract from Medicinal Herbal Plantago astiatice

Dried plantago astiatice was powdered, and 100 g of the powder was extracted with 5 volumes of water at boiling temperature for 1˜2 hrs. The water extract was passed through a Whatman #41 filter paper. The extraction procedure was repeated twice. The combined filtrate was concentrated in a rotary vacuum evaporator. The resulting concentrated extract was then precipitated by adding anhydrous ethanol. The precipitates were centrifuged and washed with anhydrous ethanol, evaporated and dried at 40° C. under vacuum yielding a crude extract. For further purification, the crude extract was resupended in H₂O and applied to diaion HP-20 column and eluted with H₂O/MeOH at 100:0 and 0:100, respectively. The MeOH eluent was collected and condensed in an evaporator (T<40° C.) and then lyophilized yielding 1.987 g of the said active extract.

### Example 2

### Preparation of the Active Extract from Medicinal Herbal Fructus Corni

Dried Fructus corni was powdered, and 100 g of the powder was soaked in absolute MeOH at a ratio of 50 ml/g, followed by refluxing (65-70° C.) for 3 hrs, and cooling twice. Any undissolved materials were removed by passage through Whatman #41 filter paper. The resulting extracts were concentrated under reduced pressure and dried yielding a crude extract. For further extraction, the crude extract was re-suspended in H₂O, partitioned sequentially with n-hexane and ethyl acetate (EtOAc) and the resulting water layer was lyophilized. The lyophilized powder was then re-suspended in H₂O and subjected to diaion HP-20SS column and eluted with H₂O/MeOH at 100:0→50:50→100:0 sequentially. The fractions of H₂O/MeOH (50:50) were collected and lyophilized yielding 3.68 g of the said active extract.

### Example 3

### Preparation of the Active Extract from Medicinal Herbal Puerariae radix

Dried Puerariae radix was powdered, and 100 g of the powder was extracted three times with 80% ethanol at room temperature for 72 hrs each time under the constant stirring condition. The combined extracts were then filtered, then concentrated and dried under reduced pressure yielding crude extract. The crude extract was re-suspended in H₂O, partitioned with ethyl acetate (EtOAc) and the resulting water layer was lyophilized. The lyophilized extract was then re-suspended in H₂O and subjected to diaion HP-20SS column and eluted with H₂O/MeOH at 100:0 and 0:100, respectively. The MeOH elutes were collected and lyophilized yielding 1.58 g of the said active extract.

### Example 4

### Preparation of the Active Extract from Medicinal Herbal Curcuma L. rhizome

The powered Curcuma L. rhizome were extracted with 100% ethanol to exhaustion under reflux at 65° C. Insoluble materials were removed by filtration via a Whatman 41 filter paper. The resulting extracts were concentrated and dried under reduced pressure yielding 1.36 g of the said active extract.

### Example 5

### Preparation of the Composite of Herbal Extracts

A composite of the extracts was obtained by mixing: 1.987 g of Plantago astiatice extract obtained from Example 1, 3.68 g of Fructus corni extract obtained from Example 2, 1.58 g of the Puerariae radix extract obtained from Example 3 and 1.36 g of Curcuma L. rhizome extract obtained from Example 4

### Example 6

### Inhibitory Effects of the Inventive Single Active Herbal Extract, Mixed Herbal Extracts on the Formation of Ages In Vitro

Glucose and Methylglyoxal induced bovine serum albumin (BSA)-AGEs formation is used to test the inhibitory effects of the inventive single herbal extract and mixed herbal extracts on the formation of AGEs in vitro.

Methods: As a protein source, BSA (Sigma-Aldrich, St. Louis, Mo., USA) was dissolved in 50 mM phosphate buffer (pH 7.4) to a concentration of 10 mg/ml. As a sugar source, a mixture of 2 mM methylglyoxal and 200 mM glucose (sigma-Aldrich, St. Louis, Mo., USA) was used. This sugar mixture was added to the prepared BSA solution for use in tests. Each active herbal extract from Plantago astiatice, Fructus corni, Puerariae radix, Curcuma L. rhizome and the herbal extract mixture prepared in Example 5 was dissolved in 10% Tween80, and this solution was added to the BSA-sugar mixture with 0.02% sodium azide as an antibacterial agent and cultured at 37° C. for 7 days. Aminoguanidine (AG), a positive control drug, was tested in the same manner except that AG was dissolved in distilled water. As a negative control group, a culture consisting of BSA, the sugar mixture and 10% Tween80 was cultured. As blanks to the control and test groups, the materials for the test group and the materials for the control group were used after preparation without incubation. Five samples for each group were used to reduce errors to the lowest possible extent. After 7 days incubation, the amounts of AGEs in test samples were determined by measuring the fluorescence at an emission wavelength of 440 nm and an excitation wavelength at 370 nm using a fluorescence Spectrophotometer. The inhibition (%) of the formation of AGEs was calculated according to the following equation:

Inhibition (%) of production=100−[(fluorescent intensity of test group−fluorescent intensity of blank to test group)/(fluorescent intensity of control group−fluorescent intensity of blank to control group)]×100.

The test results are shown in Table 1.

TABLE 1
Inhibitory effect of the single active herbal extract from Plantago
astiatice, Fructus corni, Puerariae radix, Curcuma L. rhizome and the
extract mixture prepared in Example 5 on the formation of AGEs in vitro.

| | Concentration | |
|---|---|---|
| Compounds | (mcg/ml) | Inhibition (%) |
| Extract from Plantago Astiatice | 12.5 | 18.6 ± 1.4 |
| | 25.0 | 28.3 ± 2.7 |
| | 50.0 | 48.6 ± 3.7 |
| | 100.0 | 69.3 ± 3.1 |
| Extract from Fructus Corni | 12.5 | 12.5 ± 1.9 |
| | 25 | 21.7 ± 1.7 |
| | 50 | 35.8 ± 2.9 |
| | 100 | 46.5 ± 3.6 |
| Extract from Puerariae Radix | 12.5 | 8.60 ± 4.3 |
| | 25 | 15.7 ± 3.7 |
| | 50 | 26.8 ± 4.2 |
| | 100 | 38.5 ± 3.4 |
| Extract from Curcuma L Rhizome | 12.5 | 20.2 ± 1.2 |
| | 25 | 32.1 ± 1.6 |
| | 50 | 46.5 ± 2.2 |
| | 100 | 67.8 ± 3.7 |
| Herbal extract mixture | 12.5 | 31.6 ± 3.6 |
| | 25 | 46.8 ± 4.7 |
| | 50 | 67.8 ± 3.1 |
| | 100 | 88.9 ± 3.4 |
| Aminoguanidine | 12.5 | 19.6 ± 3.6 |
| | 25 | 31.7 ± 3.5 |
| | 50 | 44.8 ± 2.9 |
| | 100 | 69.9 ± 3.5 |

As can be seen in Table 1, each single extract from Plantago astiatice, Fructus corni, Puerariae radix, Curcuma L. rhizome is effective for inhibiting the formation of AGEs at concentrations tested. The active extracts from Curcuma L. rhizome and Plantago astiatice demonstrated an inhibitory efficacy similar to positive control drug aminoguanidine at each tested concentration. Furthermore, the inhibitory activity of the herbal extract mixture at each concentration tested was superior to that of any single herbal extract and aminoguanidine, indicating that the extracts have additive effect in the inhibition of AGE formation.

### Example 7

### Effect of the Inventive Herbal Extract Mixture on the Formation and Accumulation of Ages and the Progression of Diabetic Nephropathy in Diabetic Rats

### 7.1. Test Animals and Induction of Diabetes

Male Sprague-Dawley male rats, weighing 200-250 g, aged 8-10 weeks, were used. Diabetes was induced by administration of streptozotocin (STZ, Sigma-Aldrich, USA) in 0.1 mol/L sodium citrate (pH 4.5) at a dose of 55 mg/kg intraperitoneally. After 48 hours injection, blood glucose levels of the animals were examined using glucose meter. The induction of diabetes was confirmed when the blood glucose level was greater than 300 mg/dl. Animals were maintained in diabetic conditions for 2 weeks without any treatment and blood glucose was determined every week to ensure the subsistence of diabetes. Animals with uncertain blood glucose levels or blood glucose exceeding 600 mg/dl were excluded from the study.

### (1) Treatment Protocols

The herbal extract mixture (HEM) prepared in Example 5 and aminogunidine (Sigma-Aldrich, USA), a prototype of AGE inhibitor as positive control drug, were used in the study. HEM was suspended in 1% carboxymethylcellulose (CMC) before use. Aminogunidine was dissolved in water first and then added into 1% CMC before use. The test animals were divided into the following four groups: (1) a normal age-matched control group administered with CMC (NC+CMC); (2) a diabetes-induced group administered with CMC (DC+CMC); (3) a diabetes-induced group administered with a positive control aminogunidine (DC+AG); (4) and a diabetes-induced group administered with the herbal extract mixture (DC+HEM). The diabetes-induced and the normal control groups consisted of 8 animals of each. The initial body weights and blood glucose levels of the diabetic animals were similar among the three groups. The herbal extract mixture (500 mg/kg) and aminoguanidine (50 mg mg/kg) were administered orally to the rats by gavage daily for 12 weeks. The normal control group and the diabetic control group were administered with equivalent volume of CMC daily for 12 weeks. The standard rat diet and water were available ad libitum throughout the entire treatment period.

### Results

### (2) Herbal Extract Mixture Improved the General Well-being in STZ-Diabetic Rats

The general behavioral status of the animals (i.e. locomotor activity, alertness to environmental changes) was observed every day, and the body weight and food intake were measured weekly. The beneficial effect of the inventive herbal extract mixture on the animal's general well-being was manifested after 4 week treatment evidenced by increases in body weight gain, and locomotor activity as well as the responses to their housing environment when compared with other two diabetic groups treated with aminogunidine and vehicle.

As can be seen in the Table 2 below, by the end of the experiment, the body weight increased by 143.9 g for the normal non-diabetic animals, and only 44.6 g for the diabetic group administered with CMC, and 44.0 g for the diabetic group administered with aminogunidine, whereas the body weight was increased by 78.3 g for the diabetic group treated with the herbal extract mixture, which represents a 75% (P<0.01) increase in body weight gain as compared with the other two diabetic groups treated with vehicle CMC or aminogunidine.

TABLE 2
Effect of herbal extract mixture treatment on the
body weight gain in STZ-diabetic rats.

| | Body weight (g) | Body weight (g) |
|---|---|---|
| Groups | (before treatment) | (after treatment) |
| NC + CMC | 218.6 ± 11.0 | 362.5 ± 9.3 |
| DC + CMC | 220.6 ± 10.7 | 265.2 ± 8.5** |
| DC + HEM | 216.9 ± 12.5 | 294.3 ± 7.2** |
| DC + AG | 214.6 ± 11.6 | 258.6 ± 8.1** |

Notes:
NC + CMC: normal group + carboxymethylcellulose;
DC + CMC: diabetes-induced group + carboxymethylcellulose;
DC + HEM: diabetes-induced group + herbal extract mixture;
DC + AG: diabetes-induced group + positive control aminogunidine.
All data are expressed as mean ± SD of 8 animals for each group.
**P < 0.01 versus NC + CMC group;
# p < 0.01 versus DC + CMC and DC + AG by One-way ANOVA analysis followed by Bonferroni's post hoc test.

### Herbal Extract Mixture Lowered Blood Glucose Levels in STZ-Diabetic Rats

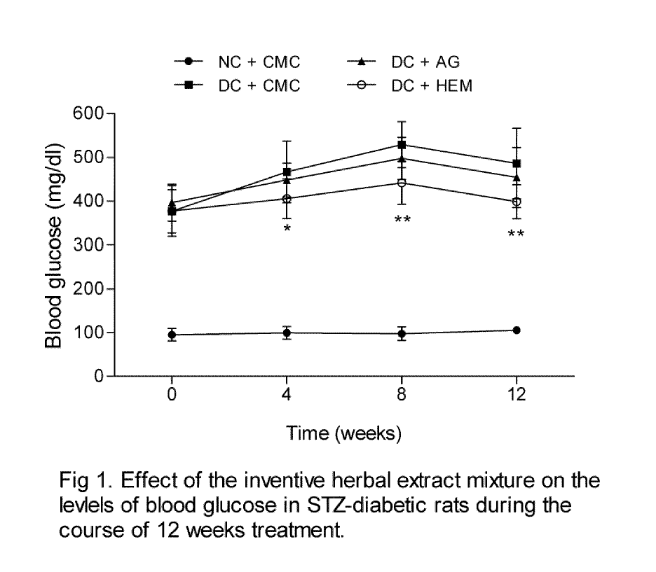

Glucose levels of tail vein blood samples were examined one day (time 0) before the treatment and measured every 2 weeks during the course of treatment using a glucometer. The blood glucose levels before and 4, 8 and 12 weeks after treatment are shown in FIG. 1.

As can be seen in FIG. 1, all STZ-induced diabetic rats had elevated blood glucose concentrations throughout the course of the study. The diabetic group treated with the inventive herbal extract mixture showed a significant decrease in the blood glucose levels at 4, (P<0.05), 8 and 12 week time points (p<0.01) as compared with the diabetic rats treated with either vehicle CMC or aminiogunidine, indicating that the herbal extract mixture has a favorable glucose-lowering activity.

Herbal extract mixture increased insulin content in the pancreatic islets and decreased the accumulation of AGEs in the pancreas of STZ-induced diabetic rats

To examine whether the inventive herbal extract mixture has protective effect on the pancreatic β-cells against toxic injury caused by STZ itself and the resulting hyperglycemia and enhanced production and accumulation of AGEs, immunohistochemistry was carried out to label the insulin in the pancreatic islets of the rats. At the end of 12 weeks treatment, rats were deeply anesthetized with sodium pentobarbital and the pancreas was removed and three pieces (3×5 mm³) of distal pancreatic tissue samples were fixed overnight in freshly prepared Zamboni's fixative. Sections (6 μm thick) were cut using a microtome and were deparaffinized, transferred into absolute ethanol and processed for immunohistochemistry. Briefly, the sections were incubated in 3% hydrogen peroxide solution to block endogenous peroxidase activity, washed with PBS buffer solution, and blocked with normal goat serum (Wuhan Boster Biological Technology Inc, Wuhan, China). The sections were then incubated with rabbit anti-rat insulin antibody (Wuhan Boster Biological Technology Inc, Wuhan, China) followed by goat anti-rabbit-Evision second antibody (Dako, USA). Sections were then visualized with DAB (Dako, USA) and washed in tap water and counterstained with hematoxylin.

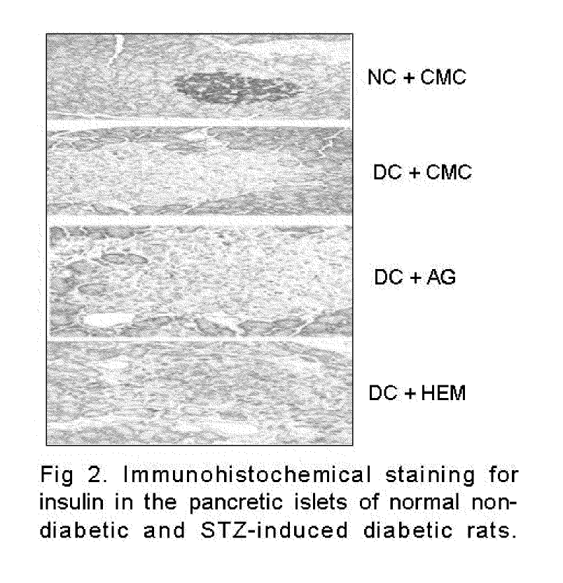

The representative micro-photos were showed in FIG. 2. As can be seen in FIG. 2, the pancreatic islets of normal non-diabetic control rats showed a normal rounded appearance and dense immunohistochemical staining for insulin. By contrast, the pancreatic islets of STZ-induced diabetic rats treated with vehicle CMC were enlarged, disorganized and fibrotic in appearance, and almost no noticeable immunoactive insulin staining. Treatment with aminogunidine for 12 weeks had no evident effect on the morphological appearance and insulin contents in the islets of the STZ-diabetic rats. However, the pancreatic islets of the STZ-diabetic rats treated with the inventive herbal mixture demonstrated a significant brown colored insulin immunoactive staining.

To examine the effect of the inventive herbal extract mixture on the formation and accumulation of AGEs, the pancreatic AGE levels were determined according to previous method [Nakayama et al., 1993, Diabetes, 345-350] with slight modifications. Minced pancreatic tissue was delipidated with chloroform and methanol (2:1,v/v) overnight. After washing, the tissue was homogenized in 0.1 N NaOH, followed by centrifugation at 8000×g for 15 min at 4° C. The amounts of AGEs in these alkali-soluble samples were determined by measuring the fluorescence at an emission wavelength of 440 nm and an excitation wavelength at 370 nm using a fluorescence Spectrophotometer. A native bovine serum albumin (BSA) preparation (1 mg/ml of 0.1 N NaOH) was used as a standard, and its fluorescence intensity was defined as one arbitrary unit (AU). The fluorescence values of samples were measured at a protein concentration of 1 mg/ml and expressed in arbitrary unit (AU) compared with a native BSA preparation. Sample protein concentrations were determined using Bradford method.

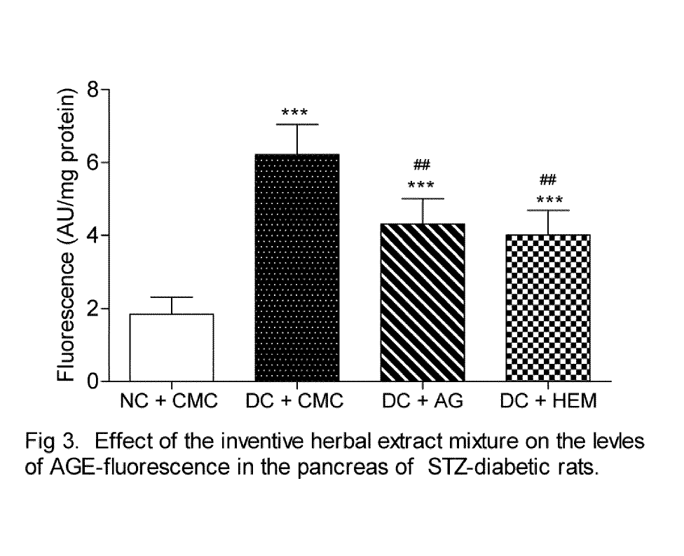

The results were demonstrated in FIG. 3. As can be seen from FIG. 3, pancreatic level of AGEs in vehicle-treated STZ-diabetic rats was significantly higher than those in nondiabetic group, but it was effectively lowered by the treatment with the inventive herbal mixture and aminogunidine.

Herbal Extract Mixture Decreased the Accumulation of Ages in the Eye Lenses of STZ-Diabetic Rats

The proteins in the lens are extremely long lived, and there is virtually no protein turnover, which provides great opportunity for glycation to occur under diabetic condition [Ranjan et al, 2006, Mol Vision 12:1077-1085]. AGEs of various derivations and molecular structures have been shown to be markedly elevated in both aged and diabetic lenses [Stitt A W, 2001, Br J Ophthamol 85:746-753; Matsumoto K et al, 1997, Biochem Biophys Res Commun 241:352-354].

To evaluate the effect of the inventive herbal extract mixture on the formation and accumulation of AGEs in the lenses of STZ-diabetic rats, at the end of the treatment, rats were deeply anesthetized with sodium pentobarbital and the eye lenses were removed and washed out the blood with ice cold PBS. Two lenses from the same animals were pooled together and were homogenized in 0.1 N NaOH. The levels of fluorescent AGEs in the lens homogenate samples were determined using a spectrofluorimeter at λₑₓ 370 nm and λₑₘ 440 nm. Results are expressed as arbitrary unit (AU)/mg of lens protein. The levels of Nε-carboxymethyl-lysine (CML), one of major known AGE structure, were measured using CML specific ELISA kit (Cell Biolabs Inc., San Diego, USA) according to the manufacturer's instruction.

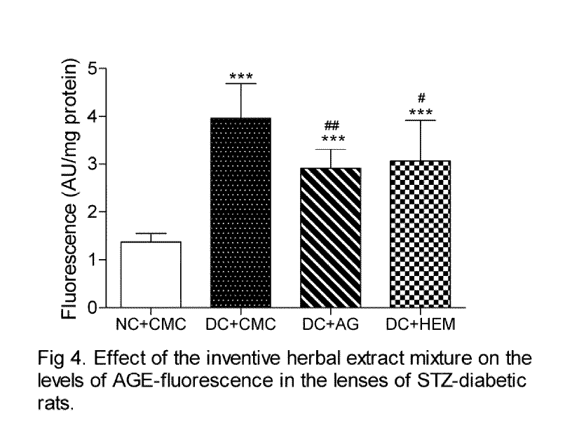

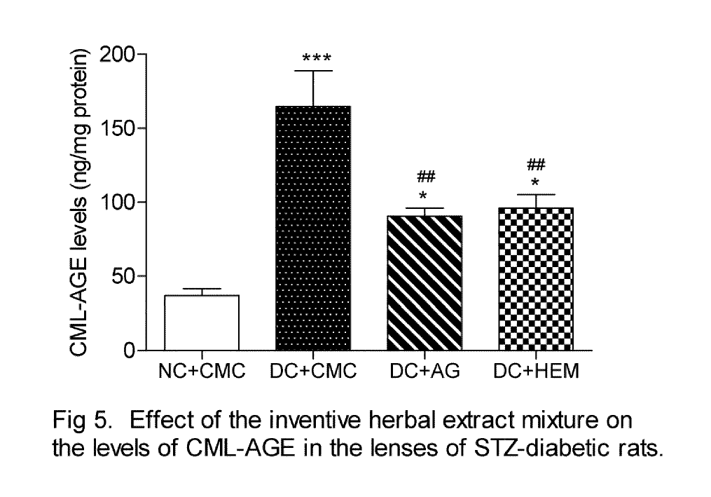

The results were shown in FIG. 4 and FIG. 5. As expected, the levels of the fluorescent AGEs and CML-AGE in the lenses of STZ-diabetic rats were elevated by 2.9 and 4.5 folds as compared with those in the normal non-diabetic rats, respectively. Administering the inventive herbal extract mixture for 12 weeks effectively reduced the production and accumulation of fluorescent AGE and CML-AGE in the diabetic lenses to the similar extent as aminogunidine, indicating that the inventive herbal mixture has the potential as an effective therapeutic for prevention and treatment of diabetic cataract and diabetic retinopathy.

### (5) Herbal Extract Mixture Reduced the Formation and Accumulation of Ages in the Descending Aorta STZ-Diabetic Rats

To evaluate the effect of the inventive herbal extract mixture on the formation and accumulation of AGEs in the macro-vasculature of STZ-diabetic rats, at the necropsy, the descending aorta was removed and washed out the blood with ice cold PBS, and were snap frozen in the liquid nitrogen and kept at −80° C. for later determination of AGE concentrations. The tissue preparation and the measurement of fluorescent AGEs and CML-AGE concentrations in the aorta were carried out in the same manner as described above for the determination of AGEs in the lenses.

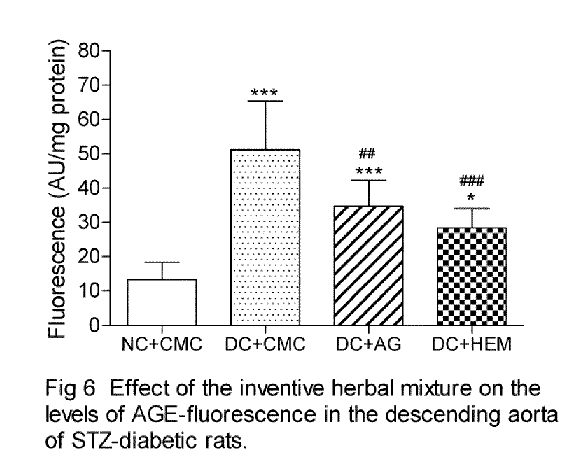

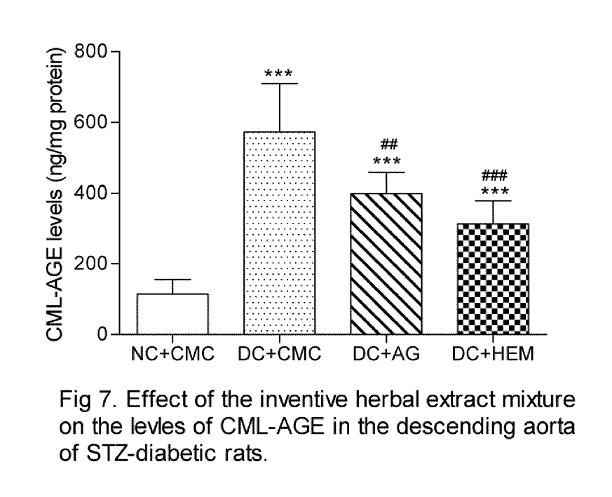

The results are shown in FIG. 6 and FIG. 7. As can be seen in FIG. 6 and FIG. 7, the descending aorta homogenates from STZ-diabetic rats had 3˜4 folds increase in the fluorescent AGEs and CML-AGE levels as compared with non-diabetic rats. Both the inventive herbal mixture and aminogunidine effectively reduced the accumulation of AGEs in the descending aorta. Accordingly, the data indicates that the inventive herb mixture possesses the potential for prevention and intervention of diabetic macro-vascular complications through inhibiting AGE accumulation of the arterial walls.

### (6) Herbal Extract Mixture Attenuated Albuminuria in STZ-Diabetic Rats

Development of albuminuria is a cardinal manifestation of glomerular injury and a pathological factor in the progression of renal dysfunction. To evaluate the protective effect of the herbal extract mixture on renal function in STZ-diabetic rats, the animals were placed in the metabolic cages and 24-hr urine samples were collected three days before the necropsy. The levels of urinary albumin were quantified using a commercial assay kit (Guangzhou WhiJa Technology, Inc. Guangzhou, China), according to the manufacturer's instructions.

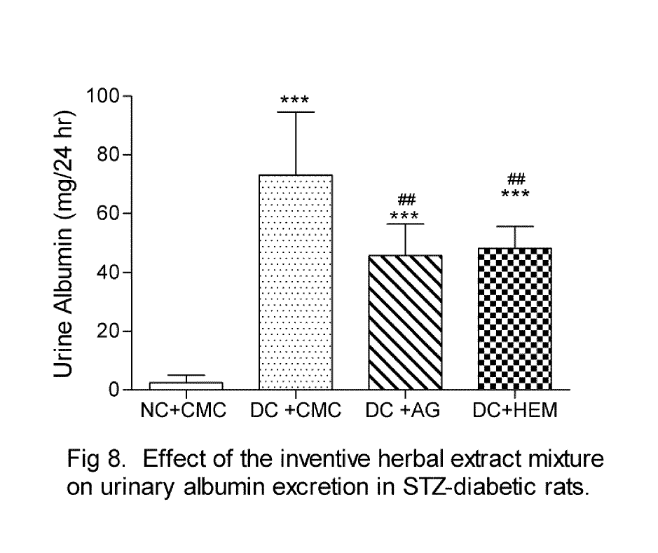

The results are shown in FIG. 8. As expected, the STZ-diabetic control rats demonstrated a heave albuminuria and the 24 hr total urinary albumin excretion was 73±21.5 mg as compared to 2.45±2.1 mg in the normal nondiabetic control group. Compared with STZ-diabetic control group, administration of the inventive herbal extract mixture and aminoguanidine reduced the 24-hr urinary albumin excretion in STZ-diabetic rats by 33.8% (P<0.01) and 37.3% (P<0.01), respectively, indicating that the inventive herbal extract mixture has renal protection effect similar to classic AGE inhibitor aminogunidine in terms of the reduction of urinary albumin excretion.

### (7) Herbal Extract Mixture Reduced Renal Age Levels in STZ-Diabetic Rats

To evaluate the AGE-inhibitory effect of the inventive herbal extract mixture in diabetic kidney, the kidneys were rapidly taken out at the necropsy and several renal cortical tissue samples (˜200-300 mg each) were collected and snap frozen in the liquid nitrogen for the determinations of AGE contents and other biomakers of interest. The tissue preparation and the measurement of fluorescent AGEs and CML-AGE concentration in the kidney cortex were carried out in the same manner as described above for the determination of AGEs in the lenses. The results are showed in Table 3 below.

TABLE 3
Effect of herbal extract mixture on the levels of AGEs in
the kidney cortex of STZ-diabetic rats

| | AGE-fluorescence | CML-AGE |
|---|---|---|
| Groups | (AU/mg protein) | (ng/mg protein) |
| NC + CMC | 2.1 ± 0.3 | 118.1 ± 35.9 |
| DC + CMC | 4.9 ± 1.0*** | 421.6 ± 126*** |
| DC + AG | 3.4 ± 0.5**# | 262.8 ± 55.4**# |
| DC + HEM | 3.1 ± 0.6*## | 240.3 ± 46.0**## |

NC + CMC: normal group + carboxymethylcellulose;
DC + CMC: diabetes-induced group + carboxymethylcellulose;
DC + AG: diabetes-induced group + positive control aminogunidine.
DC + HEM: diabetes-induced group + herbal extract mixture.
All data are expressed as mean ± SD of 8 animals for each group.
*P < 0.05,
**P < 0.01,
***P < 0.001 versus NC + CMC group;
#p < 0.05,
##P < 0.01 versus DC + CMC group by One-way ANOVA analysis followed by Bonferroni's post hoc test.

As can be seen in Table 3, the levels of fluorescent AGEs and CML-AGE were significantly elevated in the STZ-diabetic control group; administration of the inventive herbal extract mixture effectively reduced the accumulation of AGEs in the kidney of diabetic rats and the AGE-inhibitory effect of the herbal extract mixture seem to be superior to that of aminogunidine, although the difference was not statistically significant.

### (8) Herbal Extract Mixture Reduced Renal Oxidative Stress in STZ-Diabetic Rats

One of the most important consequences of “glucose toxicity” in tissue sites for diabetic complications is enhanced oxidative stress, i.e. increased production of reactive oxygen species combined with down-regulation on insufficient up-regulation of antioxidant defense mechanisms. Both hyperglycemia and increased accumulation of AGEs play an important role in the generation of reactive oxygen species. Oxidative stress has been incriminated as a major mediator in the pathogenesis of diabetic nephropathy [Baynes J W 1991, Diabetes 40: 405-412; Schnackenberg C G, 2002, Curr Opin Pharmacol 2:121-125; Kowluru Ret al 2004, J Diabetes Complications 18:282-288].

To evaluate whether the herbal extract mixture has favorable antioxidant effect on the diabetic kidney, the following biomarkers of renal oxidative stress were measured: (1) kidney cortical tissue levels of malondialdehade (MDA), which is a well-known biomarker of lipid peroxidation; and (2) the activity of superoxide dismutase (SOD), a major endogenous anti-oxidative defense enzyme.

For measurements of SOD activity and MDA contents, the renal cortical tissues were homogenized in a buffer solution (0.25 M sucrose, 0.5 mM EDTA, 50 mM HEPES, protease inhibitors) on ice and centrifuged at 4° C. at 12,000 rpm for 20 min. The supernatants were collected and analyzed with corresponding assay kits (Nanjing Jiancheng Shengwu Yanjiu Suo, Nanjing, China) according to the manufacturer's instructions,

The results are summarized in Table 4 below. As can be seen in the Table 4, the inventive HEM, but not the prototype AGE inhibitor aminogunidine, possesses potent antioxidant effect and significantly attenuated the oxidative stress evidenced by the decreased contents of MDA and increased SOD activity in the renal cortical tissue samples. Accordingly, these data indicate that except the AGE-lowering effect, the inventive herbal extract mixture has potent additional antioxidant effect, which adds values for effectively prevention and treatment of diabetic complications.

TABLE 4
Effect of herbal extract mixture on renal SOD activity and MDA
concentration in STZ-diabetic rats.

| | Renal SOD activity | Renal MDA |
|---|---|---|
| Groups | (Unit/mg protein) | (μM/mg protein) |
| NC + CMC | 3.87 ± 0.87 | 2.22 ± 0.67 |
| DC + CMC | 1.45 ± 0.64*** | 8.42 ± 2.26*** |
| DC + AG | 1.78 ± 0.42*** | 8.82 ± 1.92*** |
| DC + HEM | 2.85 ± 0.37*ab | 5.93 ± 0.52***ab |

NC + CMC: normal control group + carboxymethylcellulose;
DC + CMC: diabetes-induced group + carboxymethylcellulose;
DC + AG: diabetes-induced group + aminogunidine.
DC + HME: diabetes-induced group + herbal extract mixture.
All data are expressed as mean ± SD of 8 animals for each group.
*P < 0.05,
***P < 0.001 versus NC + CMC group;
a: p < 0.01 versus DC + CMC group;
b: P < 0.01 versus DC + AG group by One-way ANOVA analysis followed by Bonferroni's post hoc test.

### Example 8

### Test of Acute Toxicity in Oral Administration to Rats

Acute toxicity test of the inventive herbal extract mixture was conducted on six-week-old Sprague-Dawley rats. The herbal extract mixture prepared in above Examples was suspended in 1% CMC and administered by oral gavage to each test animal at a dose of 5.5 g/kg/10 ml once. After administering the test substance, the rats were observed for clinical symptoms, body weight change and death, and subjected to hematological examination and hematobiochemical examination. After autopsy, the animals were visually observed for abnormalities in abdominal and thoracic organs.

As a result, there were no special clinical symptoms in all the animals administered with the test substance. Also, there was no dead animal, and in view of the results of the body weight measurement, hematological examination, hematobiochemical examination, etc., a change in toxicity was not observed. The above results demonstrated that the inventive herbal extract mixture is a safe substance which does not show a change in toxicity up to a dose of 5.5 g/kg, which was of 100 folds higher than the intended maximal clinical dose of 4 g per day for a person of 75 kg body weight.

### Example 9

### Preparation of Tablet Formulation

A tablet formulation is shown in Table 5. All the ingredients and a suitable amount of ethanol were uniformly mixed and granulized by a wet granulation method. The granules were tabled in such a manner that one tablet weighed 500 mg.

TABLE 5
Tablet formulation of the inventive herbal extract mixture

| Ingredients | Weight (mg) |
|---|---|
| Herbal extract mixture of the invention | 250 |
| Corn starch | 75 |
| Maganesium stearate | 2 |
| Lactose | 75 |
| L-hydroxypropylcellulose | 10 |
| Polyvinylpyrrolidone | 80 |
| Sodium citrate | 8 |
| Total | 500 |

### Example 10

### Preparation of Capsule Formulation

The capsule formulation of the inventive herbal mixture consists of 250.0 mg of the medicinal herbal extracts prepared in Examples 5; 75.0 mg of corn starch; 65.0 mg of lactose; 2.0 mg of magnesium stearate and 8 mg of sodium citrate. All the components were uniformly mixed, and filled in capsules at an amount of 400 mg for each capsule.

### Example 11

### Preparation of Functional Drink

The Functional Drinks were prepared with the following compositions by a conventional processing method.

| | | |
|---|---|---|
| Herbal extract mixture of the invention | 1,000 | mg |
| Inositol | 30 | mg |
| Nicotinic acid amide | 10 | mg |
| Sodium citrate | 25 | mg |
| Pyriodoxine hydrochloride | 2 | mg |
| Water | 200 | mg |

The present invention has been described in sufficient detail with a certain degree of particularity. It is understood to those skilled in the art that the present disclosure of embodiments has been made by way of examples only and that numerous changes in the arrangement and combination of parts or elements may be resorted without departing from the spirit and scope of the invention as claimed. Accordingly, the scope of the present invention is defined by the appended claims rather than the forgoing description of embodiments.

I claim:

1. A pharmaceutical composition comprising a mixture of herbal extracts from each of Plantago astiatice L, Fructus corni, Curcuma L. rhizome, and Puerariae radix.

2. The composition of claim 1, wherein the herbal extracts from each of said Plantago astiatice L, Fructus corni, Curcuma L. rhizome, and Puerariae radix is present in an amount that is 5-85% by weight based on 100% by weight of the total mixture of extracts.

3. A functional food comprising: a mixture of herbal extracts from each of Plantago astiatice L, Fructus corni, Curcuma L. rhizome, and Puerariae radix.

4. The functional food of claim 3, wherein the herbal extracts from each of said Plantago astiatice L, Fructus corni, Curcuma L. rhizome, and Puerariae radix is present in an amount that is 5-85% by weight based on 100% by weight of the total mixture of extracts.

5. A method for inhibiting formation of advanced glycation endproducts in a subject in need thereof comprising: administering to the subject the composition or functional food of any one of claims 1 to 4.

6. A method for preventing or treating a diabetic complication or oxidative stress in a subject in need thereof comprising administering to the subject the composition or functional food of any one of claims 1 to 4.

7. The method of claim 5, wherein the diabetic complication is selected from the group consisting of diabetic nephropathy, diabetic neuropathy, and diabetic retinopathy.

8. A method of isolating herbal extracts from Plantago astiatice L, the method comprising:
boiling powdered Plantago astiatice L in an aqueous solution;
filtering and concentrating the aqueous solution; and
adding anhydrous ethanol to precipitate the herbal extracts.

9. The herbal extracts isolated by the method of claim 8.

10. A method of isolating herbal extracts from Fructus corni, the method comprising:
refluxing powdered Fructus corni in absolute methanol solution;
filtering and concentrating the solution to form a concentrate;
suspending the concentrate in water to form an aqueous solution;
washing the aqueous solution with hexane and ethyl acetate; and
lyophilizing the aqueous solution to isolate the herbal extracts.

11. The herbal extracts isolated by the method of claim 10.

12. A method of isolating herbal extracts from Curcuma L. rhizome, the method comprising:
refluxing powdered Curcuma L. rhizome in ethanol; and
filtering and concentrating the ethanol yield the herbal extracts.

13. The herbal extracts isolated by the method of claim 12.

14. A method of isolating herbal extracts from Puerariae radix, the method comprising:
extracting powered Puerariae radix with ethanol;
filtering and concentrating the ethanol to yield a concentrate;
suspending the concentrate in water to form an aqueous solution;
washing the aqueous solution with ethyl acetate; and
lyophilizing the aqueous solution to isolate the herbal extracts.

15. The herbal extracts isolated by the method of claim 14.

16. The method of any one of claim 8, 10, 12, or 14 further comprising purifying the herbal extracts on a polymeric resin column.

17. The method of claim 16 wherein the polymeric resin column is a Diaion ion exchange resin column.

18. A pharmaceutical composition or functional food comprising a mixture of herbal extracts from each of Plantago astiatice L, Fructus corni, Curcuma L. rhizome, and Puerariae radix, wherein the herbal extracts are isolated according to the methods of at least one of claims 8, 10, 12, and 14.
